(19) United States
(12) Patent Application Publication
(10) Pub. No.: US 2010/0185118 A1
(43) Pub. Date: Jul. 22, 2010

(54) BLOOD ANALYSIS DEVICE

(75) Inventors: Tetsuya Takashima, Ehime (JP); Takeshi Nishida, Fukuoka (JP)

Correspondence Address: GREENBLUM & BERNSTEIN, P.L.C., 1950 ROLAND CLARKE PLACE, RESTON, VA, 20191, US

(73) Assignee: PANASONIC CORPORATION, Osaka (JP)

(21) Appl. No.: 12/676,022

(22) PCT Filed: Sep. 4, 2008
(86) PCT No.: PCT/JP2008/002450

§ 371 Date: Mar. 2, 2010

(30) Foreign Application Priority Data
Sep. 4, 2007 (JP) 2007-228529

(51) Int. Cl. A61B 5/151

(52) U.S. Cl. 600/583

(57) ABSTRACT

A blood analysis device that can be used by one hand and has enhanced operability. The blood analysis device includes a body case (2) having a blood collection window (1), a finger cover (3) formed integrally with the body case (2) and covering a finger that faces and is in contact with the blood collection window (1), pressing means (5) for pressing the finger, which is inserted in the finger cover, toward the blood collection window (1), a recessed finger hold section (21) formed in a side face of the body case (20), and a pedestal-shaped piercing section (22) provided at the recessed finger hold section (21) and having a piercing opening (22a).

### TECHNICAL FIELD

The present invention relates to a blood analysis apparatus. For example, the present invention relates to a blood analysis apparatus that measures blood sugar levels, lactic acid levels or cholesterol levels.

### BACKGROUND ART

The blood analysis apparatus that measures blood sugar levels privately has a configuration including a casing, and in this casing, a blood sampling means and an analysis means that analyzes blood sampled by this blood sampling means. In addition, the blood analysis apparatus is very compact so that the user can easily carry the apparatus (e.g. see Patent Document 1).
Patent Document 1: Published Japanese Translation of PCT Application No. 2003-524496

### DISCLOSURE OF INVENTION

### Problems to be Solved by the Invention

However, with such a conventional blood analysis apparatus, for example, the finger of the right hand is placed in front of the blood sampling means while the casing is held by the left hand and in this state, puncturing, blood sampling and analysis operations are performed. That is, the apparatus can not be handled with only one hand because of having a configuration that needs to be used with both left and right hands and therefore it is not convenient.

The present invention solves the above-described problem, and it is therefore an object of the present invention to provide a blood analysis apparatus that can be used with one hand and improve its operability.

### Means for Solving the Problem

The blood analysis apparatus according to the present invention has a configuration including: a casing that has a blood sampling window which is an opening for sampling blood and, the casing is capable of being held in one hand; a finger cover that covers a finger facing and touching the blood sampling window; and a blood sampling section that punctures the finger inserted in the finger cover through the blood sampling window and samples the blood.

The blood analysis apparatus according to the present invention has a configuration including: a casing that has a blood sampling window which is an opening for sampling blood, and the casing is capable of being held in one hand; a finger touching section that allows a finger to face and touch the blood sampling window; a blood sampling section that punctures the finger through the blood sampling section and samples blood; and a blood sampling starting mechanism that starts the blood sampling section.

### Advantageous Effects of Invention

According to the present invention, the blood analysis apparatus has a finger cover integrally formed with the casing and covers the finger facing and touching the blood sampling window. When the finger is punctured through the blood sampling window to sample blood, the casing can be held only by inserting the finger in the finger cover. The finger for the blood sampling faces and touches the blood sampling window and therefore, in this state, blood can be sampled and analyzed. Consequently, the apparatus can be easily used with one hand, so that it is very convenient.

### BRIEF DESCRIPTION OF DRAWINGS

FIG. 1 is a perspective view of a blood analysis apparatus according to embodiment 1 of the present invention;

FIG. 2 is a perspective view of the blood analysis apparatus according to embodiment 1;

FIG. 3 is a front view explaining the usage state of the blood analysis apparatus according to embodiment 1;

FIG. 4 is a front view explaining the usage state of the blood analysis apparatus according to embodiment 1;

FIG. 5 is a front view explaining the usage state of the blood analysis apparatus according to embodiment 1;

FIG. 6 is a front view explaining the usage state of the blood analysis apparatus according to embodiment 1;

FIG. 7 is a perspective view of the blood analysis apparatus according to embodiment 2 of the present invention;

FIG. 8 is a front view of the blood analysis apparatus according to embodiment 3 of the present invention;

FIG. 9A is a front view explaining the arrangement of a finger cover of the blood analysis apparatus according to embodiment 4 of the present invention;

FIG. 9B is a front view of the blood analysis apparatus according to embodiment 4;

FIG. 10 is a perspective view of the blood analysis apparatus according to embodiment 5 of the present invention;

FIG. 11 is a front view of the blood analysis apparatus according to embodiment 6 of the present invention;

FIG. 12 is a perspective view of the blood analysis apparatus according to embodiment 6;

FIG. 13 is a perspective view of the blood analysis apparatus according to embodiment 7 of the present invention;

FIG. 14A is an enlarged view of the primary parts of a puncturing section of the blood analysis apparatus according to embodiment 7;

FIG. 14B is an enlarged view of the primary parts of a puncturing section of the blood analysis apparatus according to embodiment 7; and

FIG. 15 is a front view explaining the usage state of the blood analysis apparatus according to embodiment 7.

### BEST MODE FOR CARRYING OUT THE INVENTION

Now, embodiments of the present invention will be described in detail with reference to the drawings.

### Embodiment 1

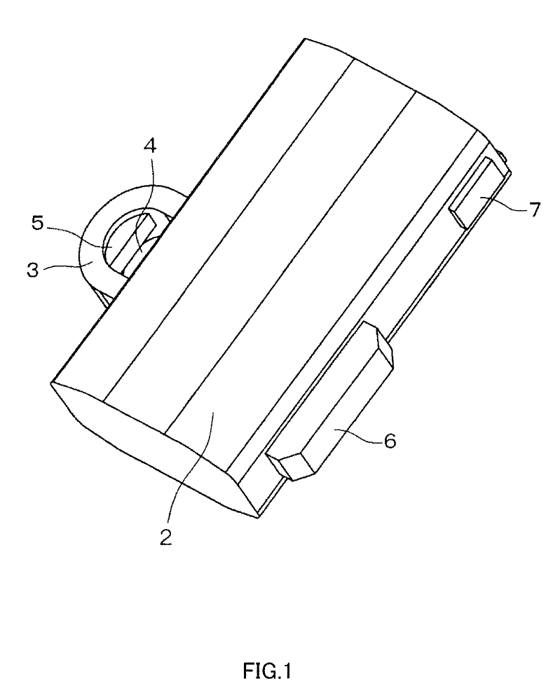

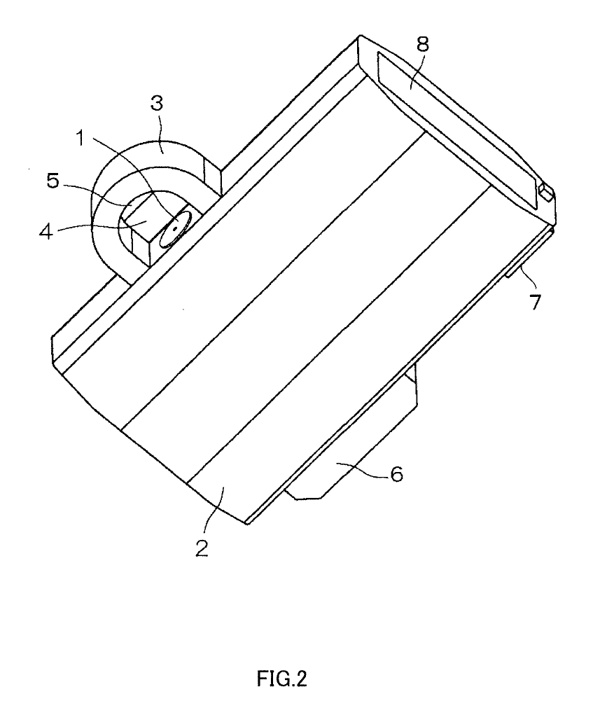

FIG. 1 and FIG. 2 are perspective views of a blood analysis apparatus according to embodiment 1 of the present invention.

In FIG. 1 and FIG. 2, the blood analysis apparatus includes an approximately rectangular-shaped casing 2 having circular blood sampling window 1. Circular blood sampling window 1 is an opening for sampling blood. In addition, casing 2 has a size allowing casing 2 to be held in one hand.

A blood sampling means (not shown) is provided in casing 2 facing blood sampling window 1. This blood sampling means has two types, which are a blood sampling means with a needle and a blood sampling means with a laser, for example. With the present embodiment, the blood sampling means with a laser is provided in casing 2.

The blood sampling means with a needle employs a puncturing system using a needle for puncturing. Meanwhile, the blood sampling means with laser light employs a puncturing system to produce laser light utilizing excitation light from a flash lamp by an Er: YAG rod and puncture with this laser light.

The finger is illuminated with laser light emitted from the blood sampling means with laser light and is punctured with laser light, and then blood is sampled. Next, the blood sampled by this blood sampling means is analyzed by an analysis means (e.g. having the configuration shown in the above-described Patent Document 1).

The present embodiment is characterized by the configuration having finger cover 3 that covers the outer circumference of the finger touching blood sampling window 1, outside casing 2 facing blood sampling window 1.

Finger cover has an approximately semicircular shape and this approximately semicircle-shaped finger cover 3 is mounted to the side surface of casing 2. In addition, finger cover 3 and casing 2 may be integrally formed. When approximately semicircle-shaped finger cover 3 is mounted to casing 2, the opening (the bowstring part of the semicircle in FIG. 2) of finger cover 3 is substantially integrated with casing 2.

Here, a space formed by casing 2 and finger cover 3 constitutes finger-inserted part 4. When the finger is inserted in finger cover 3, the inserted finger is fixed by finger cover 3.

Pushing means 5 that pushes the inserted finger toward blood sampling window 1 is provided on the inner surface of finger cover 3.

Meanwhile, pushing operation section 6 that performs the pushing operation on pushing means 5 is provided in casing 2, on the side opposite finger cover 3. Pushing operation section 6 and pushing means 5 move together, and when the user performs the pushing operation on pushing operation section 6, pushing means 5 moves toward blood sampling window 1.

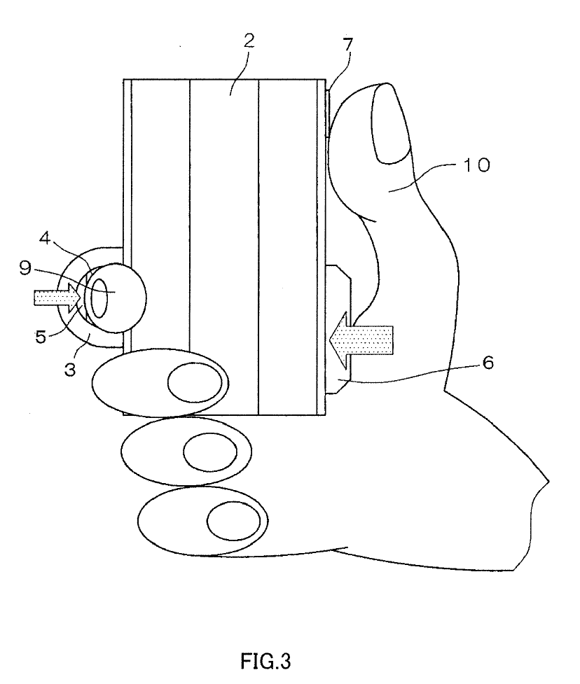

In addition, blood sampling operation section 7 is provided in casing 2, on the side opposite finger cover 3 and above pushing operation section 6 (in the upward direction in FIG. 3).

Moreover, display section 8 that displays the result of analysis by analysis means is provided on the top surface of casing 2.

FIG. 3 to FIG. 6 are front views explaining the usage state of the blood analysis apparatus according to embodiment 1 of the present invention.

FIG. 3 shows an exemplary case in which blood is sampled from index finger 9. In FIG. 3, first, index finger 9 is inserted in finger cover 3 and next, the pushing operation is performed on pushing operation section 6 with the portion around the base of thumb 10 in the palm, and therefore pushing means 5 moves toward blood sampling window 1 (in the direction indicated by the arrow of finger cover 3 in FIG. 3). By this means, index finger 9 is held and positioned appropriately in blood sampling window 1. In this state, blood sampling operation section 7 is operated by thumb 10, so that index finger 9 is illuminated with laser light generated by the blood sampling means with laser light (not shown). Then, blood is sampled from index finger 9 and then the analysis (e.g. measurement and test operations of blood sugar levels, lactic acid levels or cholesterol levels) is performed, and the result of analysis is displayed on display means 8.

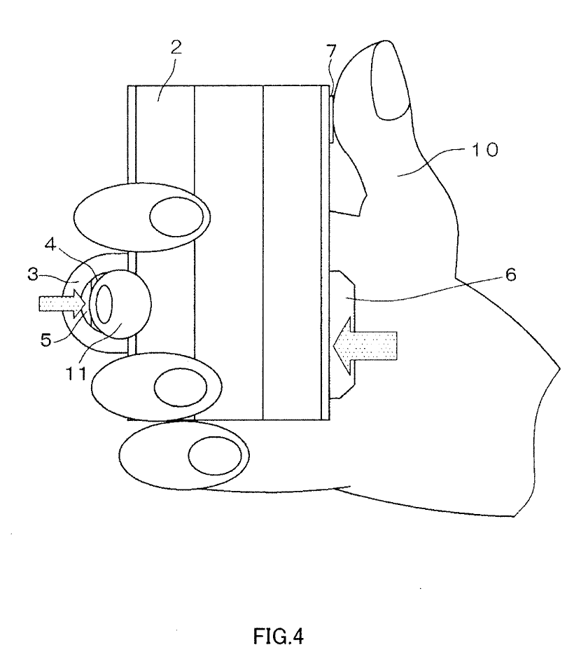

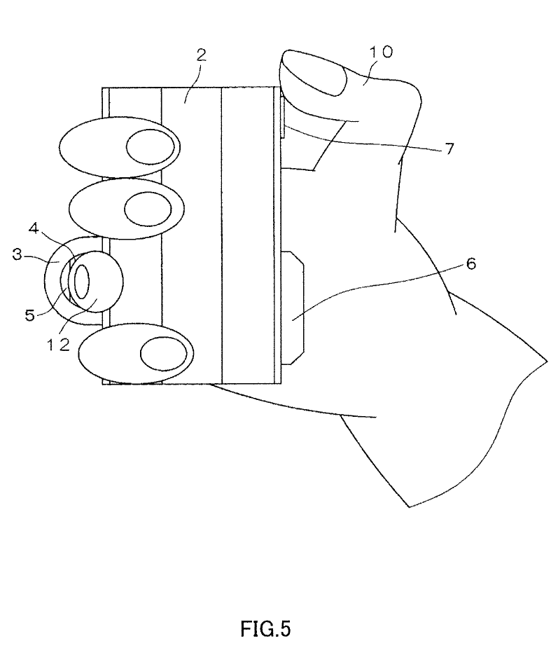

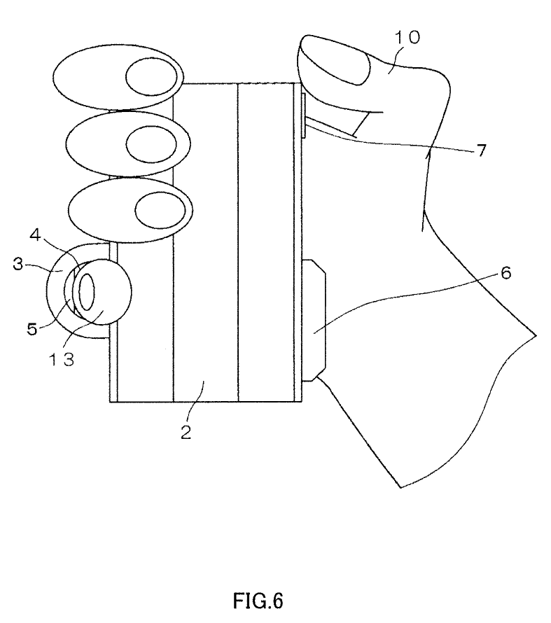

FIG. 4 shows an exemplary case where blood is sampled from middle finger 11; FIG. 5 shows an exemplary case where blood is sampled from third finger 12; and FIG. 6 shows an exemplary case where blood is sampled from little finger 13.

With those cases shown in FIG. 4 to FIG. 6, firstly the appropriate finger (any one of fingers 11 to 13) is inserted in finger cover 3, and next, the pushing operation is performed on pushing operation section 6 with the portion around the base of thumb 10 of the palm and therefore pushing means 5 moves toward blood sampling window 1 (in the direction indicated by the arrow of finger cover 3 in FIG. 4), as with the case in which blood is sampled from index finger 9 shown in FIG. 3. By this means, the appropriate finger (any one of fingers 11 to 13) can be held and positioned appropriately in blood sampling window 1. In this state, blood sampling operation section 7 is operated by thumb 10, so that the appropriate finger (any one of fingers 11 to 13) is illuminated with laser light. Then, blood exuding from the appropriate finger (any one of fingers 11 to 13) is sampled and then the analysis (e.g. measurement and test operations of blood sugar levels, lactic acid levels or cholesterol levels) is performed and the result of analysis is displayed on display means 8.

As described above, according to the blood analysis apparatus of the present embodiment, casing 2 can be held only by inserting the finger in finger cover 3 and then blood sampling and analysis can be performed in this state because the finger used for the blood sampling faces and touches blood sampling window 1. That is, casing 2 can be easily held and then the blood sampling and the analysis can be performed only by one hand and therefore the handiness of the apparatus can be significantly improved.

In addition, when the blood is sampled, the appropriate finger (any one of fingers 9, 11 to 13) might come away by reflex action (so-called finger escape) in the direction to part from blood sampling window 1 by the impact caused by illuminating the finger (any one of fingers 9, 11 to 13) with laser light. With the present embodiment, however pushing means 5 is pushed toward blood sampling window 1 by the operation of pushing operation section 6 and therefore the appropriate finger (any one of fingers 9, 11 to 13) is held and positioned appropriately in blood sampling window 1. By this means, the so-called finger escape does not occur and therefore the blood sampling by illuminating with laser light can be reliably performed.

Moreover, with the present embodiment 1, the pushing force of pushing means 5 toward blood sampling window 1 can be adjusted by the operation of pushing operation section 6, so that the apparatus can be held optimally regardless of the size of the finger (any one of fingers 9, 11 to 13) and therefore the blood sampling can be performed stably.

Here, the adjustment of the pushing force of pushing means 5 toward blood sampling window 1 by operating pushing operation section 6 as described above can be easily performed utilizing an air pressure. Specifically, pushing operation section 6 and pushing means 5 are connected by an air flow passage (not shown) and pushing operation section 6 is pushed strongly toward casing 2, so that a large amount of the air flows in the back side of pushing means 5 and makes pushing means 5 expand as an air bag and therefore pushing means 5 pushes the finger (any one of fingers 9, 11 to 13) strongly toward casing 2.

In addition, pressing means 5 stops applying the pressing force to the finger (any one of fingers 9, 11 to 13) by stopping pushing pushing operation section 6, so that the finger (any one of fingers 9, 11 to 13) can be easily removed from finger cover 3.

Here, although an example of performing the blood sampling and the analysis by the right hand has been explained in the above-described FIG. 3 to FIG. 6, the puncturing, the blood sampling and the analysis can be easily performed by the left hand in the same way.

In addition, with the present embodiment 1, the blood sampling means with laser light that performs puncturing and blood sampling by generating laser light is provided in casing 2 as a blood sampling means. The finger is illuminated and punctured with laser light emitted from the blood sampling means with laser light through blood sampling window 1, and blood is sampled by the illuminating and puncturing operations. In this case, even if blood sampling operation section 7 is operated erroneously despite the state where the finger is not inserted in finger cover 3, laser light illuminates finger cover 3 but does not leak outside of finger cover 3, so that the safety is improved.

As described above, according to the present embodiment, the blood analysis apparatus includes casing 2 having blood sampling window 1 and finger cover 3 that is integrally formed with casing 2 and that covers the finger facing and touching blood sampling window 1, so that casing 2 can be held only by inserting the finger in finger-inserted section 4. In addition, the appropriate finger can be held and positioned appropriately in the part of blood sampling window 1. Moreover, since the finger used for the blood sampling faces the blood sampling window 1 and is defined and therefore the blood sampling and the analysis can be performed in this state. By this means, the apparatus can be used with one hand very easily, so that the handiness can be significantly improved. An excellent effect that the apparatus can be easily and reliably used by a person inexperienced in the blood test or a sight-impaired person can be obtained.

In addition, with the present embodiment 1, pushing means 5 that pushes the inserted finger toward blood sampling window 1 is provided in finger cover 3, and pushing operation section 6 that operates this pressing means 5 is provided opposite finger cover 3 of casing 2. As a result of this, so-called finger escape in the blood sampling can be prevented and therefore the blood sampling by illuminating with laser light can be reliably performed.

Moreover, with the present embodiment 1, if the finger does not contact finger cover 3 when blood sampling is performed, finger cover 3 can block laser light, so that the safety can be more improved.

In addition, with the present embodiment 1, finger cover 3 is substantially integrated with casing 2, so that the overall apparatus is compact and convenient to carry the apparatus.

### Embodiment 2

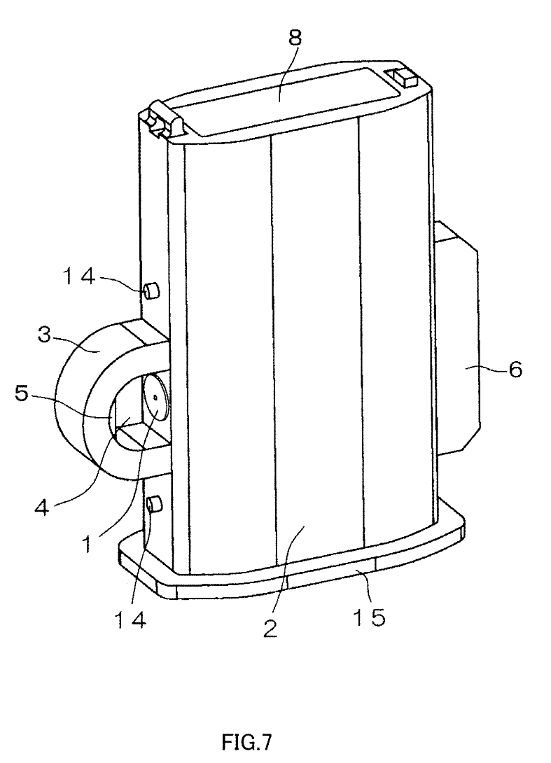

FIG. 7 is a perspective view of the blood analysis apparatus according to embodiment 2 of the present invention. The same components as in FIG. 1 and FIG. 2 are assigned the same reference numerals and the overlapping description will be omitted.

In FIG. 7, the blood analysis apparatus includes at least one or more blood sampling operation sections 14 on the same plane as on finger cover 3 of casing 2 and in the vicinity of finger cover 3. In this case, blood sampling sections 14 are provided in both sides (two positions in the upper and lower part in FIG. 7) of finger cover 3 and therefore blood sampling operation sections 14 can be easily pressed by the fingers other than the finger used for the blood sampling even if the apparatus is operated with one hand.

Although, in embodiment 1, blood sampling operation section 7 operated by thumb 10 is provided above pushing operation section 6, blood sampling operation sections 14 are provided on the side surface of casing 2 to which finger cover 3 is mounted in the present embodiment 2, instead of blood sampling operation section 7.

In addition, stand flange 15 is provided on the bottom surface of casing 2 in order to prevent casing 2 from falling.

According to the present embodiment, another finger (any one of fingers 9, 11 to 13) instead of thumb 10 can operate blood sampling operation sections 14.

### Embodiment 3

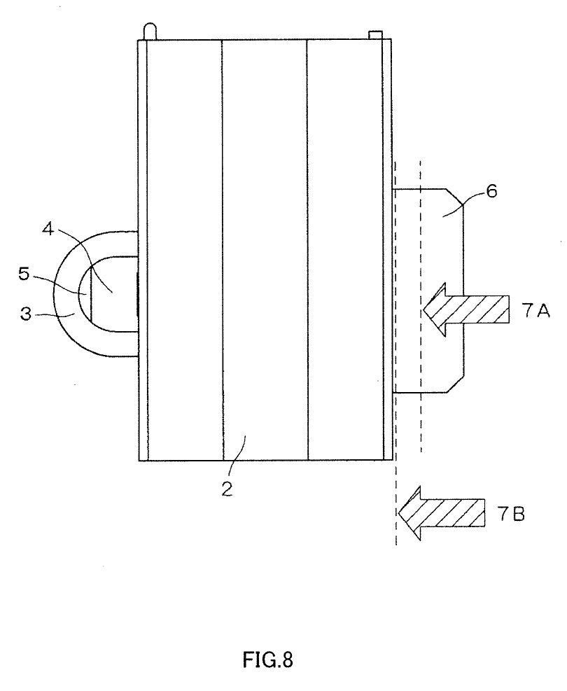

FIG. 8 is a front view of the blood analysis apparatus according to embodiment 3 of the present invention. The same components as in FIG. 1 and FIG. 2 are assigned the same reference numerals and the overlapping description will be omitted.

In FIG. 8, the blood analysis apparatus includes pushing operation section 6 having a two-step pushing formation. In this two-step pushing formation, pushing means 5 operates in first step 7A and the blood sampling operation means operates in second step 7B.

### Embodiment 4

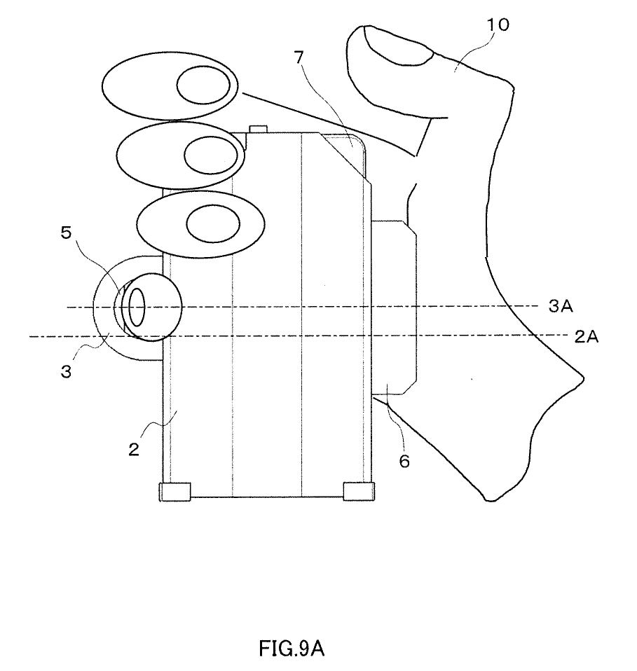

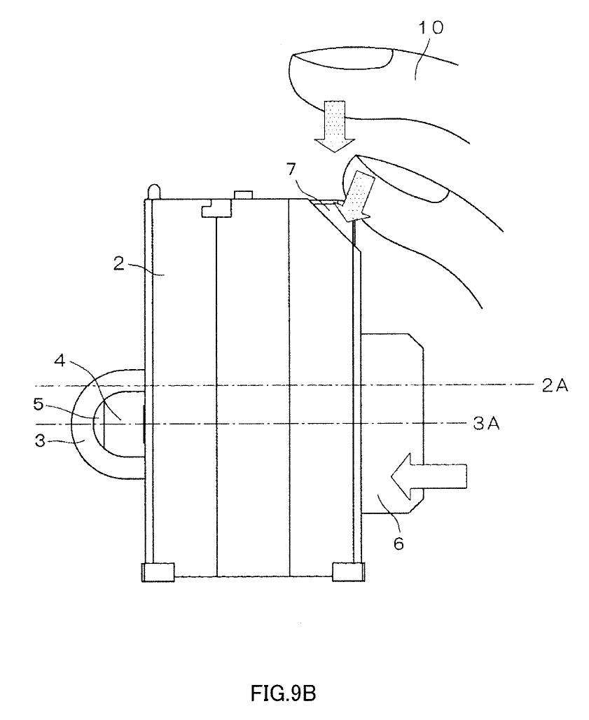

FIG. 9A and FIG. 9B are front views of the blood analysis apparatus according to embodiment 4 of the present invention. The same components as in FIG. 1 and FIG. 2 are assigned the same reference numerals and the overlapping description will be omitted.

FIG. 9A is a drawing explaining the arrangement of finger cover 3 in comparison with FIG. 9B. FIG. 9A shows a case where the little finger is inserted in finger cover 3 to perform a puncturing operation. In the case of FIG. 9A, center line 3A of finger cover 3 is located above (the upper direction in FIG. 9A) center line 2A of casing 2.

Thus, in FIG. 9A, when the user attempts to press blood sampling operation section 7, the position of thumb 10 is apart from blood sampling operation section 7, so that it is difficult to operate blood sampling operation section 7. That is, this configuration is very hard to operate.

In order to solve the above-described defect, as shown in FIG. 9B, center line 3A of finger cover 3 is located on the same line as center line 2A of casing 2 or located below center line 2A (the lower direction in FIG. 9B) in the present embodiment as shown in FIG. 9B.

In FIG. 9B, the blood analysis apparatus has a configuration such that, when the pushing operation is performed on pushing operation section 6 with the base of thumb 10 of the palm, this pushing operation section 6 projects from casing 2 outward.

In addition, finger cover 3 is mounted such that center line 3A of finger cover 3 in the vertical direction is located below center line 2A of casing 2.

According to the present embodiment, pushing operation section 6 projects from casing 2 outside, so that subsequently blood sampling operation section 7 operated by thumb 10 can be very easily performed. In addition, finger cover 3 is mounted in a position lower than center line 2A of casing 2 in the vertical direction, so that the blood sampling operation by each finger as shown in FIG. 3 to FIG. 6 can be easily performed.

### Embodiment 5

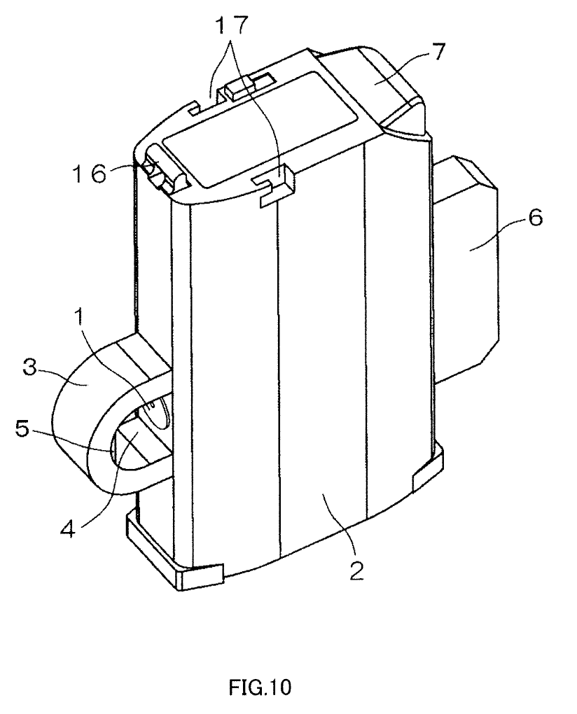

FIG. 10 is a perspective view of the blood analysis apparatus according to embodiment 5 of the present invention. The components the same as in FIG. 9A and FIG. 9B are assigned the same reference numerals and the overlapping description will be omitted.

In FIG. 10, the blood analysis apparatus has strap attachment section 16 and strap supporting grooves 17 above finger cover 3 of casing 2 (the upper direction in FIG. 10).

A strap (not shown) is attached to strap attachment section 16, and if this strap is unnecessary, a part of the strap is engaged with strap supporting grooves 17.

### Embodiment 6

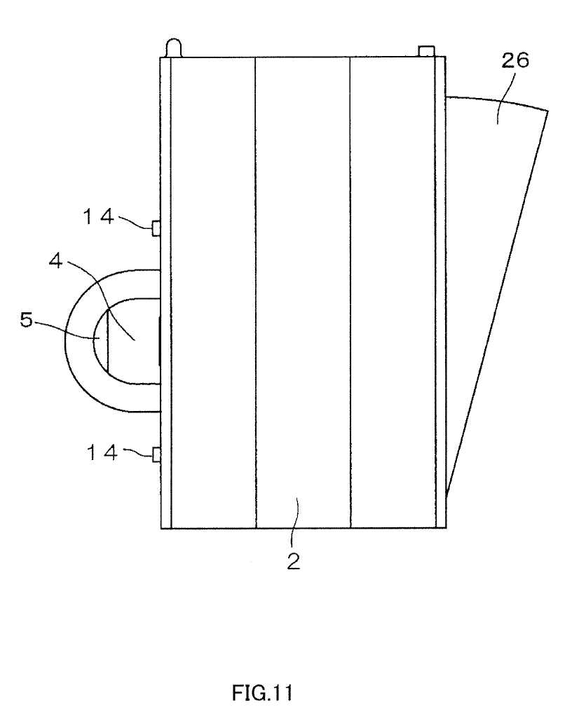

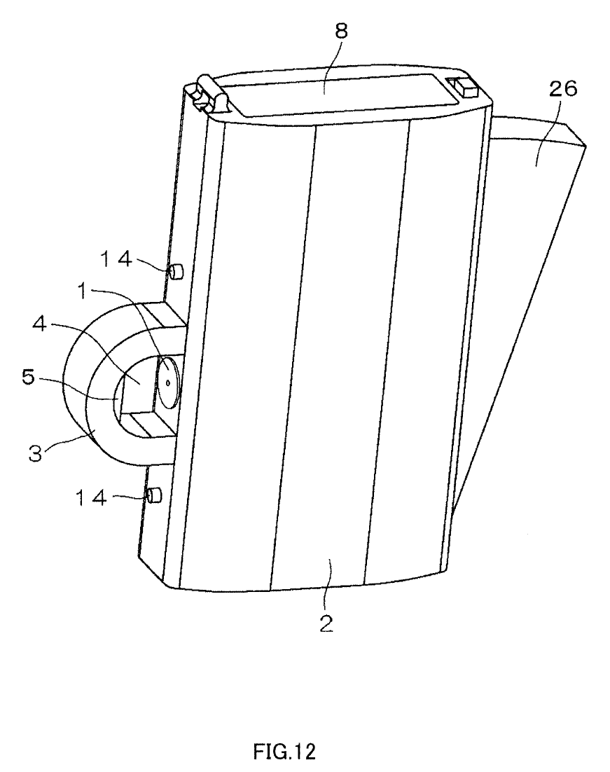

FIG. 11 is a front view of the blood analysis apparatus according to embodiment 6, and FIG. 12 is a perspective view of the blood analysis apparatus according to embodiment 6. The same components as in FIG. 7 are assigned the same reference numerals and the overlapping description will be omitted.

In FIG. 11 and FIG. 12, the blood analysis apparatus has pivotable pushing operation section 26 that has the central axis of rotation on the lower end of casing 2, that is supported on the lower end of casing 2 and that pivotably moves in the upper part of casing 2. Pushing operation section 26 and pushing means 5 moves together, and when the user performs the pushing operation on this pushing operation section 26, that is, the user pushes pushing operation section 26 into the casing, pushing means 5 can be pushed out casing 2.

When the apparatus is not used, the upper end of pushing operation section 26 moves to casing 2 and therefore is housed in casing 2, so that there is no projection. In this state, pushing operation section 26 is held by a clip and so forth (not shown). At this time, pushing means 5 is maximally pushed out toward casing 2, so that the finger (any one of fingers 9, 11 to 13) cannot be inserted in finger cover 3.

In the use of the apparatus, when the above-described clip that holds pushing operation section 26 housed in casing 2 is removed, pushing operation section 26 is placed in a state such that the upper end of pushing operation section 26 rotates from casing 2 and maximally parts from casing 2 as shown in FIG. 11 and FIG. 12, so that pushing means 5 provided in finger cover 3, which moves together with this pushing operation section 6, is hardly pushed out toward casing 2. By this means, the finger (any one of fingers 9, 11 to 13) can be easily inserted in the finger cover.

At this time, the holding and positioning force on the finger (any one of fingers 9, 11 to 13), which is applied by pushing means 5, is determined based on the amount of pushing by pushing operation section 26 toward casing 2. In this state where the finger is inserted in finger cover 3, blood sampling operation sections 14 provided above and below finger cover 3, respectively (upper and lower directions in FIG. 11 and 12) are operated and therefore the blood sampling and the analysis can be performed.

At this time, all blood sampling operation sections 14 do not always have to be operated and one of blood sampling operation sections 14 is enough.

### Embodiment 7

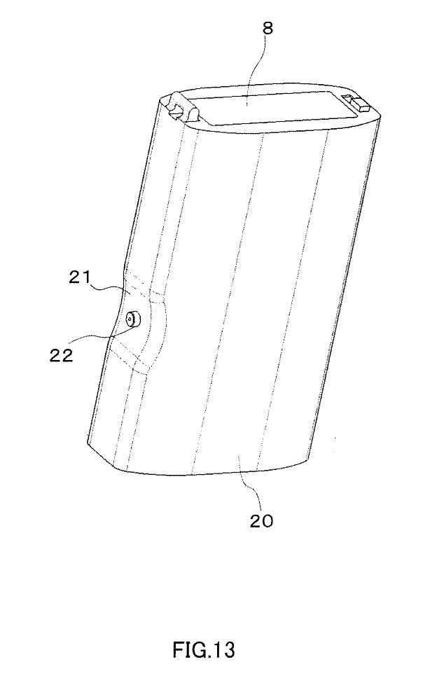

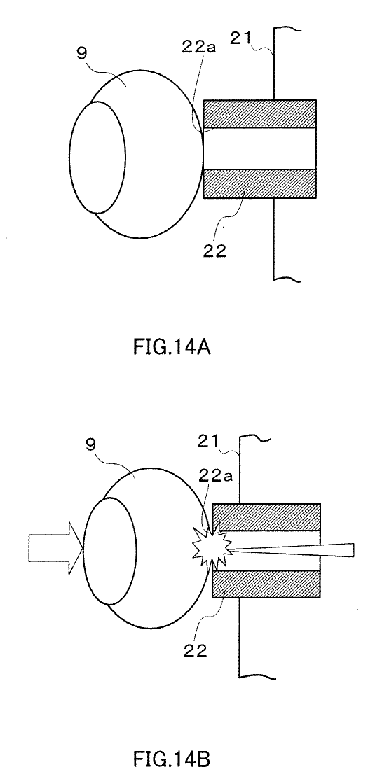

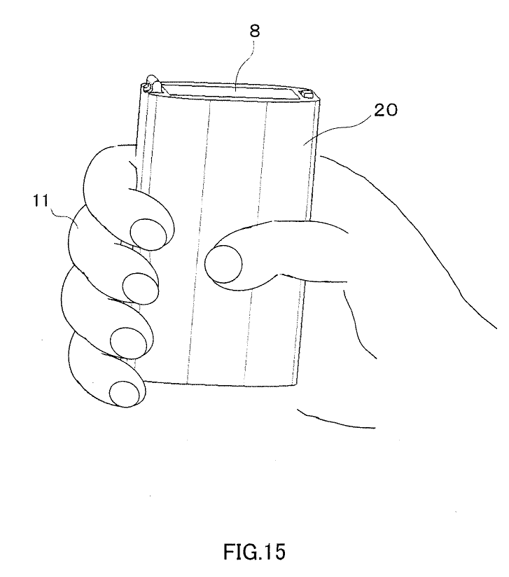

FIG. 13 is a perspective view of the blood analysis apparatus according to embodiment 7; FIG. 14A and FIG. 14B are enlarged views of the primary parts of a puncturing section of the blood analysis apparatus; and FIG. 15 is a front view explaining the usage state of the blood analysis apparatus. The components the same as in FIG. 7 are assigned the same reference numerals and the overlapping description will be omitted.

In FIG. 13, the blood analysis apparatus includes casing 20. Casing 20 is provided with finger touching section 21. Finger touching section 21 is provided with puncturing section 22 also serving as a puncturing switch. That is, in embodiment 7, finger touching section 21 and puncturing section 22 are provided instead of finger cover 3 and blood sampling window 1 of the blood analysis apparatus of each above-described embodiment (embodiments 1 to 6). In addition, pushing means 5 provided inside finger cover 3 and pushing operation section 6 provided in casing 2 are omitted in embodiment 7. Moreover, display means 8 that displays the result of analysis by the analysis means is provided on the top surface of casing 20 in embodiment 7.

Finger touching section 21 is a concave part provided on the side surface of casing 20. When casing 20 is held in one hand, the finger to be punctured is easily fitted in finger touching section 21 because finger touching section 21 has a curved shape.

Puncturing section 22 is a pedestal having a puncturing opening that is provided to project from finger touching section 21.

In addition, a blood sampling means with laser light (not shown) is housed in casing 20, and laser light emitted from this blood sampling means with laser light penetrates puncturing section 22 and punctures skin (e.g. index finger 9, and hereinafter referred to as “skin 9”).

As shown in FIG. 14A, skin 9 such as a finger of the patient touches puncturing opening 22a of puncturing section 22.

In this state in which skin 9 touches puncturing opening 22a, puncturing section 2 is pressed as shown in FIG. 14B. Laser light is emitted from the blood sampling means with laser light in casing 20, and this laser light penetrates puncturing opening 22a of puncturing section 22 and is illuminated. By this means, a small amount of blood exudes from skin 9.

As shown in FIG. 15, casing 20 has a rectangular solid shape and is sized to be held in one hand. When the user attempts to perform a puncturing operation, the user places the finger (e.g. middle finger 11) on finger touching section 21 and grips casing 20 in his/her hand. Puncturing section 22 projecting from the surface of finger touching section 21 is pressed into casing 20 by gripping casing 20 in one hand, and the puncturing and the blood sampling are performed.

In addition, since puncturing section 22 projects from the surface of the concave part of finger touching section 21, an appropriate focal distance of laser light can be obtained and the location in which the puncturing is performed can be clearly indicated to the user.

Moreover, since finger cover 3 does not project from casing 20 as with in each embodiment described above, the apparatus is more compact and is convenient to carry the apparatus.

As described above, according to the present embodiment, the blood analysis apparatus has concave-shaped finger touching section 21 provided on the side surface of casing 20 and pedestal-shaped puncturing section 22 having puncturing opening 22a in this concave-shaped finger touching section 21, so that the blood sampling and the analysis can be performed only by touching finger touching section 21 with the finger and pushing puncturing section 22. In this case, the user may only touch finger touching section 21, which is a concave part, with the appropriate finger and grip casing 20 lightly. By this means, the apparatus can be used very easily with one hand and the handiness can be significantly improved. An excellent effect that the apparatus can be easily and reliably used by a person inexperienced in the blood test or a sight-impaired person, is obtained.

In addition, in the present embodiment, finger touching section 21 is provided with puncturing section 22, and the blood sampling starting mechanism is not operated until the finger in contact with finger toughing section 21 presses puncturing section 22, so that the safety can be assured.

Here, as the above-described blood sampling starting mechanism, blood sampling operation section 7 may be provided opposite finger touching section 21 in casing 20 as with blood sampling operation section 7 described in the above-described embodiment 1.

In addition, as the blood sampling starting mechanism, blood sampling operation sections 14 may be provided in the same side as in finger touching section 21 or in the vicinity of finger touching section 21 in casing 20 as with blood sampling operation sections 14 described in the above-described embodiment 2 or embodiment 6.

Moreover, in the present embodiment, since there is no component projecting from casing 20, the overall apparatus can be more compact.

The above description is illustration of the preferred embodiments of the present invention and the scope of the invention is not limited to this.

Moreover, although the name “blood analysis apparatus” is used in each above-described embodiment for convenience of explanation, it goes without saying that the name of the apparatus may be a “blood test apparatus”, a “puncturing apparatus”, a “body fluid measurement apparatus” and so forth.

Moreover, for each component constituting the above-described blood analysis apparatus, such as the kind of cartridge, the number and the connection method thereof are not limited. Particularly, the finger cover may have any shape. For example, a configuration such that a part of the finger cover is cut out and therefore the finger cover does not cover the entire circumference of the finger may be applicable. In addition, preferably, the finger cover has a configuration allowing the inserted finger to be fixed adequately when the finger is inserted in the finger cover. From this viewpoint, the finger cover may be referred to as “finger fixing section”.

The disclosure of Japanese Patent Application No. 2007-228529, filed on Sep. 4, 2007, including the specification, drawings and abstract, is incorporated herein by reference in its entirety.

### INDUSTRIAL APPLICABILITY

The present invention is applicable to a blood analysis apparatus that samples blood from skin by a blood sampling means such as a laser emitting device and analyzes components of the blood. The present invention is also applicable to a blood analysis apparatus and so forth that measure blood sugar levels, lactic acid levels and cholesterol levels.

1. A blood analysis apparatus comprising:
a casing that has a blood sampling window which is an opening for sampling blood, and the casing is capable of being held in one hand;
a finger cover that covers a finger facing and touching the blood sampling window; and
a blood sampling section that punctures the finger inserted in the finger cover through the blood sampling window and samples the blood.

2. A blood analysis apparatus comprising:
a casing that has a blood sampling window which is an opening for sampling blood, and the casing is capable of being held in one hand;
a finger touching section that allows a finger to face and touch the blood sampling window;
a blood sampling section that punctures the finger through the blood sampling section and samples blood; and
a blood sampling starting mechanism that starts the blood sampling section.

3. The blood analysis apparatus according to claim 1, wherein the finger cover is integrally formed with the casing.

4. The blood analysis apparatus according to claim 1, wherein a center of the finger cover is provided below a center line of the casing in a vertical direction.

5. The blood analysis apparatus according to claim 1, wherein:
the blood sampling section is a laser type that generates laser light and performs a puncturing operation and a blood sampling operation; and
the finger cover blocked the laser light generated by the laser type, if the finger does not touch the finger cover.

6. The blood analysis apparatus according to claim 1, wherein the finger cover has a pushing section that pushes an inserted finger toward the blood sampling window.

7. The blood analysis apparatus according to claim 6, further comprising a pushing operation section that is provided opposite the finger cover and that operates the pushing section.

8. The blood analysis apparatus according to claim 1, further comprising a blood sampling operation section that is provided opposite the blood sampling window and that operates the blood sampling section.

9. The blood analysis apparatus according to claim 1, further comprising at least one or more blood sampling operation sections that are provided in the same side as in the blood sampling window and in the vicinity of the blood sampling window, and that operate the blood sampling section.

10. The blood analysis apparatus according to claim 7, wherein the pushing operation section projects from the casing outside at least in a pushing operation.

11. The blood analysis apparatus according to claim 7, wherein the pushing operation section rotates about an axis provided at one end of the casing.

12. The blood analysis apparatus according to claim 7, wherein the pushing operation section has at least a two-step pushing formation, and the pushing section operates in a first step and the blood sampling section operates in a second step.

13. The blood analysis apparatus according to claim 1, further comprising a display section that displays a result of analysis of the blood sampled by the blood sampling section.

14. The blood analysis apparatus according to claim 1, wherein the casing has a strap attachment section.

15. The blood analysis apparatus according to claim 2, further comprising a blood sampling operation section that is provided opposite the blood sampling window and that operates the blood sampling section.

16. The blood analysis apparatus according to claim 2, further comprising at least one or more blood sampling operation sections that are provided in the same side as in the blood sampling window and in the vicinity of the blood sampling window, and that operate the blood sampling section.

17. The blood analysis apparatus according to claim 2, wherein the blood sampling section is a laser emitting device that punctures skin with laser light in a noncontact state.

18. The blood analysis apparatus according to claim 2, wherein the finger touching section is a concave part of the casing.

19. The blood analysis apparatus according to claim 2, wherein:
the blood sampling section is formed by a pedestal having a blood sampling opening; and
the blood sampling starting mechanism is performed by pressing the pedestal.

20. The blood analysis apparatus according to claim 2, wherein the blood sampling starting mechanism is enabled when the finger in contact with the finger pushing section is pressed.
